(19) United States
(12) Patent Application Publication
(10) Pub. No.: US 2006/0177530 A1
(43) Pub. Date: Aug. 10, 2006

(54) Method of treating diabetes type II

(76) Inventor: Roberto Crea, San Mateo, CA (US)

Correspondence Address: THE LUTHER LAW FIRM, 12198 E. COLUMBINE DR., SCOTTSDALE, AZ, 85259, US

(21) Appl. No.: 10/564,924

(22) PCT Filed: Jul. 14, 2004
(86) PCT No.: PCT/US04/22889

§ 371 Date: Jan. 17, 2006

Related U.S. Application Data
(60) Provisional application No. 60/487,011, filed on Jul. 14, 2003.

(51) Int. Cl. A61K 36/63; A61K 31/05

(52) U.S. Cl. 424/769; 514/731

(57) ABSTRACT

A method of treating early diabetic neuropathy includes administering a composition comprising an aqueous extract of olives is disclosed. A method of treating patients with Cfiber neuropathy includes administering a composition comprising an aqueous extract of olives. A method of treating patients with large fiber and C-fiber neuropathy includes administering a composition comprising an aqueous extract of olives. Preferably the aqueous extract of olives contains a weight ratio of hydroxytyrosol to oleuropein of between about 1:1 and about 400:1. More preferably, the weight ratio is between about 3:1 and about 200:1. Most preferably, the weight ratio is between about 5:1 and about 50:1. Alternately, an extract from grape leaves can be used to treat patients with large fiber and Cfiber neuropathy.

### BACKGROUND

1. Technical Field

This invention is in the field of medical therapy, more specifically in the field of treatment of symptoms of diabetes type II.

2. Prior Art

Type II diabetes is a type of adult onset diabetes. A patient in the early phase of diabetic nerve damage has early diabetic neuropathy (EDN). Some researchers have estimated the prevalence of neuropathies in EDN at about 10%. This may be unjustifiably low, as that statistic was based only on a clinical presentation of large sensory fiber disease (e.g., diminished nerve conduction values and quantitative sensory deficits of vibration and touch). Among pre-diabetic and early post-diabetic subjects, most demonstrated a disorder of small somatic sensory nerves, which tended to be generalized to upper and lower limbs in contrast to the large fiber disease that originates in the lower limbs (Herman R et al., Society Neurosci Abstr. 2000). These sensory nerves are defined as unmyelinated (C-fibers) and poorly myelinated (A-delta) neurons. The C-fibers are derived from polymodal receptors which convey signals of pain secondary to noxious heat, chemicals (e.g., capsaicin), and proton build up, as well as signals to vasodilate the microcirculation (e.g., pre-capillary arterioles) (Holzer P, CAPSAICIN IN THE STUDY OF PAIN, Acad Press 9:191-210, 1993). EDN therefore can lead to microcirculatory impairment, i.e., failure to vasodilate (Herman, ibid.) which commonly leads to retinopathy, nephropathy, and worsening neuropathy. Small sensory fiber dysfunction may lead to relative vasoconstriction with failure to vasodilate appropriately with heat and chemical stimuli. Further, such impairment leads to a rise in threshold and magnitude of pain. Alteration in C-fiber function raises the risk of pressure ulceration of the skin and difficulty in healing wounds. Such occurrences are usually associated with a cascade of events threatening the span and quality of life of the individual.

Another small fiber system may be dysfunctional in early EDN, namely, the sympathetic nervous system (SNS). Although a rise in catecholamines may or may not occur in this subject population, autonomic SNS neuropathy may be present (Ziegler et al, Exp Clin Endocrinol Diabetes 107:421-430, 1999). Potentially such a neuropathy would alter the basic vasoconstrictor tone of the peripheral vasculature and control of the heart rate. Autonomic neuropathy may also affect heart rate variability (reduction) and power spectrum (low frequency) responses. The impairment of post-ganglionic sympathetic neurons and/or their pre-synaptic receptors may lead to some alleviation of vasoconstrictor tone (due to failure of small sensory fibers to vasodilate). However, plastic changes, i.e., up-regulation of post-synaptic receptors, may lead to further vasoconstriction of blood vessels.

What is needed is a method for treating EDN, preferably with a natural product.

### SUMMARY OF THE INVENTION

In one embodiment, a method of treating early diabetic neuropathy includes administering a composition comprising an aqueous extract of olives. Preferably the aqueous extract of olives contains a weight ratio of hydroxytyrosol to oleuropein of between about 1:1 and about 400:1. More preferably the aqueous extract of olives contains a weight ratio of hydroxytyrosol to oleuropein of between about 3:1 and about 200:1. Most preferably the aqueous extract of olives contains a weight ratio of hydroxytyrosol to tyrosol of between about 5:1 and about 50:1.

In another embodiment, a method of treating patients with C-fiber neuropathy includes administering an aqueous extract of olives. Preferably the aqueous extract of olives contains a weight ratio of hydroxytyrosol to oleuropein of between about 1:1 and about 400:1. More preferably the aqueous extract of olives contains a weight ratio of hydroxytyrosol to oleuropein of between about 3:1 and about 200:1. Most preferably the aqueous extract of olives contains a weight ratio of hydroxytyrosol to tyrosol of between about 5:1 and about 50:1.

In yet another embodiment, a method of treating patients with large fiber and C-fiber neuropathy includes administering an aqueous extract of olives. Preferably the aqueous extract of olives contains a weight ratio of hydroxytyrosol to oleuropein of between about 1:1 and about 400:1. More preferably the aqueous extract of olives contains a weight ratio of hydroxytyrosol to oleuropein of between about 3:1 and about 200:1. Most preferably the aqueous extract of olives contains a weight ratio of hydroxytyrosol to tyrosol of between about 5:1 and about 50:1.

In yet another embodiment, there is provided a method of treating patients with large fiber and C-fiber neuropathy that includes administering an extract of olive leaves.

### DETAILED DESCRIPTION OF A PREFERRED EMBODIMENT

Natural antioxidants present in high concentration in olives continue to capture the interest of medical science. There is a growing body of evidence revealing the unique antioxidant activity of a family of compounds, polyphenols, against oxygen-derived free radicals in pathological processes. It is now clear that the health benefits of extra virgin olive oil in the promotion of healthy breast tissue, colon function, cardiovascular function and other health states associated with oxidative stress can be attributed to the strong antioxidant activity of polyphenols. These antioxidants may also help maintain overall health and wellness through their anti-bacterial and anti-viral activity.

Specifically oleuropein and hydroxytyrosol are the natural polyphenols from olives which provide the highest level of free radical protection ever reported for any natural antioxidant compound.

There is a patented two-step process to utilize olive water rich in antioxidant polyphenols. First, the pits are removed from the olives. Next the pitted olives are pressed, which removes both water and oil from the olives. Then the polyphenolic-rich water is separated from the oil and processed to avoid air oxidation and to release the highest quantity of antioxidant activity. Olive water from the olive oil milling process, or vegetation water, thus becomes an economical and yet previously unused source of natural antioxidant polyphenols (including hydroxytyrosol). Two patents, U.S. Pat. Nos. 6,165,475 and 6,197,308, describe this process in detail and are hereby incorporated by reference. Oleuropein also is commercially obtained from olive leaves; it can be used instead of or added to the aqueous extract of olives.

The vegetation water can be acidified to between pH of 2.0 and 4.0 to convert oleuropein to hydroxytyrosol (U.S. Pat. No. 6,165,475). U.S. Pat. No. 6,416,808, hereby incorporated by reference, discloses additional ways to purify for hydroxytyrosol, including aqueous-alcoholic extraction. The weight ratio of hydroxytyrosol to oleuropein is preferably between 1:1 and 400:1, more preferably between about 3:1 and about 200:1 and most preferably between about 5:1 and 100:1. The extracts may also be formulated to contain various weight ratios of hydroxytyrosol and tyrosol of between about 10:1 and about 50:1, and preferably between about 15:1 and about 30:1.

The polyphenols can be administered orally or parenterally. Oral dosage forms can be in a solid or liquid form. Such dosage forms can be formulated from purified polyphenols or they can be formulated from aqueous or aqueous-alcoholic extracts. Regarding the latter, aqueous or aqueous-alcoholic (e.g., water-methanol or water-ethanol) extracts can be spray-dried to provide a dry powder that can be formulated into oral dosage forms with other pharmaceutically acceptable carriers.

The solid oral dosage form compositions are prepared in a manner well known in the pharmaceutical arts, and comprise polyphenols in combination with at least one pharmaceutically acceptable carrier. In making such compositions, polyphenols, either in substantially pure form or as a component of a raw distillate or extract, are usually mixed, diluted or enclosed with a carrier. The carrier can be in solid form, semi-solid or liquid material which acts as a vehicle, carrier or medium for the active ingredient. Alternatively, the carrier can be in the form of a capsule or other container to facilitate oral administration. Thus, the solid oral dosage forms for administration in accordance with the present invention can be in the form of tablets, pills, powders, or soft or hard gelatin capsules.

Polyphenols can be formulated with other common pharmaceutically-acceptable excipients, including lactose, dextrose, sucrose, sorbitol, mannitol, starches, gums, calcium silicate, microcrystalline cellulose, polyvinylpyrrolidone, methyl cellulose, water, alcohol and the like. The formulations can additionally include lubricating agents such as talc, magnesium stearate and mineral oil, wetting agents, emulsifying and suspending agents, preserving agents such as methyl- and propylhydroxybenzoates, sweetening agents or flavoring agents. Further, the polyphenols can be formulated to provide quick, sustained or delayed release of the active ingredient after administration to a subject.

Alternatively, the polyphenols can be in liquid form wherein the pharmaceutically acceptable carrier is water or an aqueous-alcoholic (e.g., ethanol) medium. Parenteral formulations of polyphenols are prepared using standard techniques in the art. They are commonly prepared as sterile injectable solutions, using a parenterally acceptable carrier such as isotonic saline prior to administration to a subject.

Safety and toxicity studies at 0.5, 1 and 2 g/kg of HT and other phenolic compounds indicate no clinical or pathological toxicity in mice. The clinical dose of HT is in the range of 1-2000 mg/day, more preferably in the range of 5-500 mg/day and most preferably 10-100 mg/day.

From the foregoing, it can be seen how various objects and features of the invention are met. Those skilled in the art can now appreciate from the foregoing description that the broad teachings of the present invention can be implemented in a variety of forms. Therefore, while this invention has been described in connection with particular embodiments and examples thereof, the true scope of the invention should not be so limited. Various changes and modifications may be made without departing from the scope of the invention, as defined by the appended claims. The following example illustrates a method of using HT in accordance with the invention. The example is intended to illustrate, but in no way limit, the scope of the invention.

### EXAMPLE 1

### Clinical Study

To measure the antioxidant properties of olive polyphenols (OL) in subjects with non-symptomatic EDN by testing specific biochemicals related to oxidative status (lipid peroxidation), endogenous antioxidant pool, inflammation and nitrous oxide metabolism and bioactivity. Another purpose of the study is to assess the potential therapeutic effect of polyphenols on EDN, such as on small sensory neuropathy, on small and large sensory nerve fiber neuropathy and cutaneous microcirculation.

The study design is a double-blind, placebo (PL) controlled, randomized, parallel study of OL and PL (two tablets in both AM and PM) in two populations of non-symptomatic EDN: Group A has small sensory nerve fiber disorder (OL=15; PL=15) and Group B has small and large sensory fiber disease (OL=15; PL=15). All dependent variables will be obtained at baseline and at one, three and six months post-therapy.

Patients will be screened with respect to the inclusion/exclusion criteria and assignment to Group A or B by applying a modified Quantitative Sensory Test (see below) (Perkins et al., Diabetes Care 24:250-256,2001). Possible inclusion criteria are as follows:
1. The patient must be capable of giving informed consent.
2. The patient must be willing and able to attend all study visits and complete all tests.
3. The patient must be between the ages of 30 and 65.
4. The patient will have been diagnosed with Type 2 diabetes mellitus for less than 5 years.
5. The patient's diabetes is treated with diet, exercise and/or oral antidiabetic agents.
6. The patient must have a Body Mass Index (BMI) of 22-32, thereby including some pathologically obese subjects.
7. The patient must have evidence of small fiber neurologic disease alone or of both small and large fiber disease.
8. The patient's neuropathy must be asymptomatic.
9. The patient's glycated hemoglobin is ≦8.0.

Possible exclusion criteria are as follows:
1. The patient takes insulin to control diabetes.
2. The patient has smoked within the previous two years.
3. The patient has taken vitamins, minerals, antioxidants, or herbal supplements within the previous thirty days.
4. The patient consumes more than 2 oz. of olive oil daily.
5. The patient has neuropathy of non-diabetic origin.
6. The patient has a history of alcohol abuse.
7. The patient has end-stage renal disease.
8. The patient has had significant exposure to neurotoxins (e.g., heavy metals, fertilizers, solvents).
9. The patient has HIV.
10. The patient has any of the following: thyroid disease, myocardial infarction within the past 3 months, congestive heart failure, angina, hepatic disease, or significant neurological diseases.
11. The patient has a current infection.
12. The patient has an open ulcer or wound.
13. The patient has peripheral vascular disease (non-palpable pedal pulses, intermittent claudication, rest pain, or an ankle-brachial index <7.0.
14. The patient has been admitted to the hospital in the past for mental illness or is currently under treatment for severe mental illness.
15. The patient is participating in another study.
16. The patient has been diagnosed with cancer (except non-melanoma skin cancer).
17. The patient is pregnant, lactating, or of childbearing age without an acceptable form of birth control.
18. The patient suffers from chronic pain and takes medication to control it.
19. The patient has an autoimmune disease (connective tissue or vasculitis).
20. The patient takes any of the following medications: nonsteroidal anti-inflammatory drugs (including colchicines), narcotic analgesics, antidepressants, prednisone, phenyloin, or calcium blockers.

Also at the initial interview, the patient will receive a short and modified-version of quantitative sensory testing (QST for large and small fiber function by examination of psycho-physical responses to vibratory, tactile and noxious heat stimuli to both feet. A patient is assigned to Group A or B if one or both feet reveal a neuropathy.

Upon qualified patients, the following microcirculatory and small sensory nerve assessment are performed:

C-fibers are tested with noxious heat and pepper (capsaicin) solution. Noxious heat is achieved by the use of a brass contact thermode, 1.5 cm in diameter, enclosed in a plastic collar (Moor Instruments, Devon, England). The temperature of the thermode is raised from 32° C. to 44° C. at a rate of 2° C./sec and maintained at 44° C. for 20 minutes. The collar contains an aperture for a laser Doppler probe in the center of the brass contact (for “direct” blood flow) and an aperture 100 mm from the edge of the contact for another probe (for “indirect” blood flow).

Among normal subjects, the pain threshold is about 43-45° C. All perceive a verv hot sensation at about 44° C. Among diabetics, there is often an absence of appreciation of hot and/or pain. As C-fibers convey heat pain signals to the central nervous system, this suggests that diabetics experience a C-fiber disorder. The perception of hot and/or pain adapts when the temperature is maintained at 44° C. Conventionally, the temperature is maintained for 20 minutes to assess the effect of heat on blood flow. In pre-diabetics and early diabetics, direct blood flow is usually markedly reduced. Indirect flow also is impaired.

The topical application of 1.0% capsaicin (CAP) as a mixture of CAP dissolved in 75% absolute ethanol and 25% saline solution, is infused into a 300 μl plastic Perspex containing both direct and indirect blood flow monitoring probes for 45 minutes. Normally, CAP induces discomfort and marked cutaneous direct and indirect blood flow. In pre-diabetics and early diabetics, CAP does not induce discomfort and induces lower levels of blood flow, both suggesting C-fiber dysfunction as CAP preferentially stimulates C-fibers in the skin.

Endothelial-dependent and -independent vasodilitation is measured by iontophoresis. Iontophoresis is a method of transferring a drug into or across the skin non-invasively through the use of a low-voltage direct current. Ions migrate between oppositely charged electrodes, facilitating their transport through the intervening skin. Since iontophoresis works via differences in electrical charge, the drug being delivered must be composed of both negative and positive ions. The ions must also be small enough to pass through the tight junctions of the epidermis.

Iontophoresed substances are dissolved in a control vehicle (e.g., methylcellulose, 2% in solution with deionized water), which also is used to detect the microcirculatory changes associated with the iontophoretic current. In this manner, current-induced changes in the microcirculation can be measured directly and later subtracted from the total effects of a drug to remove the influence of current from the final analysis of drug effect. The active substances include acetylcholine (Ach; 10%; positively charged), which is an endothelium-dependent vasodilator and sodium nitroprusside (SNP; 1%; negatively charged), which is an endothelium-independent vasodilator. These are administered with a Perspex plastic dispenser (registered trademark of Ineos Acrylics, UK) using a continuous serial dose escalation paradigm of 0.2 mA for 5, 10, 20, 40, 80 and 160 seconds, with a 180-second interval between each stimulus. The paradigm is used to obtain a set of characteristic dose-response curves within and between subjects. The dose equivalents are represented as millicoulombs (i.e., mA×seconds=mC).

The sympathetic nervous system, or adrenergic receptor, activation is also measured by iontophoresis, of norepinephrine (0.1%; positively charged) for 300 seconds and a post-response period of 20 minutes. In normal subjects, this dose produces vasocontriction at the site of stimulation and vasodilatation at a removed site (i.e., 1 cm from the stimulus area), suggesting that norepinephrine causes neurogenic vasodilatation via C-fiber stimulation. This was evidenced when a local anesthetic blocked this action.

Values obtained from laser Doppler flowmetry are recorded during each stimulation period. Average flow ratings for the direct and indirect responses are tabulated for the baseline (60 seconds) pre-stimulus phase. For each stimulation, the peak blood flow response is recorded, i.e., during the 20 min heat, 30 min CAP, and at each dose/post-dose period of the iontophoretic protocol. The average baseline values are subtracted from the peak responses to yield an absolute value for the stimulus. Moreover, with the iontophoresis protocol, the effect of the vehicle and current must be subtracted from the absolute value to yield a definitive flow rate for the substance administered.

Cardiac rate (R—R interval) and power spectral analysis of the rate will be performed using a Polar S-810 heart rate monitor. A reduction in R—R variability and shift in power spectrum is indicative of autonomic dysfunction.

Quantitative sensory testing (QST) is used to evaluate changes in large-fiber neuropathy, including the following two assessments. Semmes-Weinstein Monofilament Esthesiometer (SWME) provides a simple, reproducible calibrated means of assessing protective sensation and has been used to estimate the risk of ulceration. In addition to its use as a prognostic tool for the prediction of foot complications, it has been established as a screening tool for the diagnosis of large-fiber neuropathy. SWME is conducted using a 5.07/10 gm monofilament applied to a noncallused area on the plantar aspect of the great toe. The application will be repeated four times on both feet in a nonrhythmic manner by an independent examiner who will record the number of correct responses out of a possible 8 on a scoring sheet. Failure to perceive 5 or more applications establishes the presence of large-fiber neuropathy. Vibratory testing (VT) using an on-off method has also been validated as a screening tool for large-fiber neuropathy. Prior to testing with each testing modality, a reference sensation is established by applying the stimulus to the sternum. The patient is then asked to close the eyes and describe the sensations experienced by each modality.

Blood and/or urine tests will be performed. Upon admission, at least some of the following tests can be done: glucose, BUN, creatinine, electrolytes, calcium, hepatic function, lipid panel, cholesterol, triglycerides, LDL, HDL (and ratio), homocysteine, folate, serum B12, serum magnesium and serum zinc. Other tests include hemoglobin Alc, microalbumin, overnight glucose tolerance test (OGTT, blood glucose and insulin), oxidative status (blood and urine F2 isoprostanos), endogenous antioxidant activity (TAC, glutathione (GSH), glutathione peroxidase), inflammation (C-reactive protein), and nitrous oxide metabolism and bioactivity (urinary nitrites and nitrates; cGMP). These will be compared with the values from 50 normal individuals.

The nutrition-focused portion of the physical examination comprises history and examination. The patient is first questioned about age, sex, medical and nursing problem lists over the last 24 hours, weight/anthropometric, dietary/alimentation, biochemical, clinical, drug/medication profile and other variables that impact on nutriologic status. For the examination, the following equipment is needed: portable lighted magnifier, 128 Hz tuning fork, sterile tongue blades and data gathering forms. Vibration sense is determined by the criteria of Fuller, i.e., a three-point scale with no sense of vibration, mild sense of vibration and strong sense of vibration. For each patient, the eyes, everted eye lid, eyebrows, lips, tongue, gums, and teeth are inspected. The tongue is lightly palpated with the sterile tongue blade. As examination dictates, the practitioner expands the exam to inspection of scalp, hair, skin and nails. Additional history, if indicated, includes weight change, normal eating pattern/changes, food intolerances, appetite, change in elimination, feeding status, hydration and absorptive status.

The daily dose is 20 mg of HT within 1200 mg of polyphenols. Control patients receive placebo medication. The study lasts six weeks.

Significant improvement in the functions of C-fiber, large fiber, sympathetic nerve disease or microcirculation are indicators that the polyphenol treatment is effective.

In light of the detailed description of the invention and the example presented above, it can be appreciated that the several aspects of the invention are achieved. It is to be understood that the present invention has been described in detail by way of illustration and example in order to acquaint others skilled in the art with the invention, its principles, and its practical application. Further, the specific embodiments of the present invention as set forth are not intended as being exhaustive or limiting of the invention, and that many alternatives, modifications, and variations will be apparent to those skilled in the art in light of the foregoing example and detailed description. Accordingly, this invention is intended to embrace all such alternatives, modifications, and variations that fall within the spirit and scope of the following claims. While some of the examples and description above include some conclusions about the way the invention may function, the inventor does not intend to be bound by those conclusions and functions but puts them forth only as possible explanations.

What is claimed is:

1. A method of treating early diabetic neuropathy comprising administering a composition comprising an aqueous extract of olives.

2. The method of claim 1 wherein the aqueous extract of olives contains a weight ratio of hydroxytyrosol to oleuropein of between about 1:1 and about 400:1.

3. The method of claim 1 wherein the aqueous extract of olives contains a weight ratio of hydroxytyrosol to oleuropein of between about 3:1 and about 200:1.

4. The method of claim 1 wherein the aqueous extract of olives contains a weight ratio of hydroxytyrosol to tyrosol of between about 5:1 and about 50:1.

5. A method of treating patients with C-fiber neuropathy comprising administering an aqueous extract of olives.

6. The method of claim 5 wherein the aqueous extract of olives contains a weight ratio of hydroxytyrosol to oleuropein of between about 1:1 and about 400:1.

7. The method of claim 5 wherein the aqueous extract of olives contains a weight ratio of hydroxytyrosol to oleuropein of between about 3:1 and about 200:1.

8. The method of claim 5 wherein the aqueous extract of olives contains a weight ratio of hydroxytyrosol to tyrosol of between about 5:1 and about 50:1.

9. A method of treating patients with large fiber and C-fiber neuropathy comprising administering an aqueous extract of olives.

10. The method of claim 9 wherein the aqueous extract of olives contains a weight ratio of hydroxytyrosol to oleuropein of between about 1:1 and about 400:1.

11. The method of claim 9 wherein the aqueous extract of olives contains a weight ratio of hydroxytyrosol to oleuropein of between about 3:1 and about 200:1.

12. The method of claim 9 wherein the aqueous extract of olives contains a weight ratio of hydroxytyrosol to tyrosol of between about 5:1 and about 50:1.

13. A method of treating patients with large fiber and C-fiber neuropathy comprising administering an extract of olive leaves.

14. The method of claim 13 wherein the extract of olive leaves is blended with an aqueous extract of olives, with hydroxytyrosol or a combination thereof.
